(19) United States
(12) Patent Application Publication
(10) Pub. No.: US 2023/0230663 A1
(43) Pub. Date: Jul. 20, 2023

(54) SERVERS, SYSTEMS, AND METHODS FOR MODELING THE CARBON FOOTPRINT OF AN INDUSTRIAL PROCESS

(71) Applicant: AVEVA SOFTWARE, LLC, Lake Forest, CA (US)

(72) Inventors: Ketrina Katragjini Prifti, Valencia (ES); Mohammad Shahid Amin, Reading (GB); Zeljko Patljak, Glasgow (GB)

(21) Appl. No.: 18/096,173

(22) Filed: Jan. 12, 2023

Related U.S. Application Data
(60) Provisional application No. 63/298,723, filed on Jan. 12, 2022.

(51) Int. Cl. G16C 20/30; G06Q 10/0631; G06Q 50/04; G06Q 30/018; G16C 20/70

(52) U.S. Cl. CPC G16C 20/30; G06Q 10/06315; G06Q 30/018; G06Q 50/04; G16C 20/70

(57) ABSTRACT

In some embodiments, the disclosure is directed to a system that predicts the carbon footprint of an industrial process. In some embodiments, the system is configured to monitor the amount of energy used in one or more process steps in an industrial process. In some embodiments, the system is configured to determine a carbon intensity for each of the one or more process steps. In some embodiments, the system is configured to generate a report including the carbon intensity. In some embodiments, the system is configured to determine the effect different raw material have on each of the one or more processing steps. In some embodiments, the system is configured to generate an optimum blend of raw materials that reduces the carbon intensity of one or more steps. In some embodiments, the system is configured to generate a blend of source fuels that reduces the industrial facilities overall carbon footprint.

### CROSS-REFERENCE TO RELATED APPLICATION

This application claims the benefit of and priority to U.S. Provisional Application No. 63/298,723, filed Jan. 12, 2022, which is incorporated herein by reference in its entirety.

### BACKGROUND

Environmental regulations and risks of climate change are pressuring the refinery industry to minimize its greenhouse gas (GHG) emissions. Many Oil & Gas companies have joined the campaign to significantly reduce their net carbon footprint by 2050 by developing new strategies, new business models, and alternative renewable fuel technologies. Major companies have set a new goal to become net zero companies by 2050 or sooner, and to help the world get to net zero.

The problem with greenhouse gas emissions is twofold. There is the use aspect, where the refined fuel is burned in automobiles and factories, and then there is the refining aspect, where fuel is needed to refine the crude oil into different fuels such as kerosene and gasoline. A problem that currently exists in the refining industry is that the amount of energy needed to refine the crude material into a finished product is variable. The variability is due in part to different energy requirements for different crude types. For example, heavy crude takes more energy to refine than light crude. Due to different chemical composition, the crude material from one geographic region may take more energy to refine than crude material from another region. Further complicating the issue, the amount of energy required at specific refinement steps vary for both the type and source of crude materials. When these different subsets of crude material are mixed together during refinement, prediction of total carbon intensity for refinement becomes even more complex.

While minimizing an industrial facility’s carbon footprint is a priority, so is minimizing cost. While buying a cheaper one crude material may seem advantageous, the cost of the energy to refine it may drive up the total cost of the final fuel product higher than if a more expensive crude material was purchased for the same volume of output. However, predicting when to purchase the refining energy at its lowest cost may mitigate the cost issue.

Therefore, there is a need in the art for a system that can identify the carbon intensity needed to refine different types crude materials and crude material blends into a final product. In addition, there is a need in the art for a system to determine the lowest energy cost for different crude types and blends.

### SUMMARY

Some embodiments of this disclosure are directed to systems and methods for optimizing the amount of energy required to convert a raw material (e.g., a crude material such as oil) into a final product. In some embodiments, the crude material includes oil. In some embodiments, the final product includes fuel (e.g., gasoline, diesel, jet fuel, etc.)

In some embodiments, a system for controlling a carbon footprint of an industrial process comprises one or more computers comprising one or more processors and one or more non-transitory computer readable media. In some embodiments, the one or more non-transitory computer readable media includes program instructions stored thereon that when executed cause the one or more computers to execute one or more program steps. In some embodiments, a program step includes instructions to receive, by the one or more processors, raw material data. In some embodiments, the raw material data comprises one or more of raw material location data, raw material type data, raw material blend data, and raw material property data for each of one or more raw materials.

In some embodiments, a program step includes instructions to monitor, by the one or more processors, one or more sensors configured to determine fuel source consumption data comprising an amount of one or more fuel sources required by one or more process steps in the industrial process. In some embodiments, a program step includes instructions to determine, by the one or more processors, the amount of one or more fuel sources required by one or more process steps in the industrial process to process each of the one or more raw materials. In some embodiments, a program step includes instructions to receive, by the one or more processors, fuel source data comprising fuel emissions data for the one or more fuel sources. In some embodiments, a program step includes instructions to execute, by the one or more processors, a carbon intensity analysis configured to output a carbon intensity value for at least one of the one or more process steps based on the fuel emissions data. In some embodiments, a program step includes instructions to generate, by the one or more processors, a carbon intensity report comprising the carbon intensity value. In some embodiments, a program step includes instructions to display, by the one or more processors, the carbon intensity report on one or more graphical user interfaces (GUIs).

In some embodiments, the fuel emissions data comprises an amount of CO₂ emitted per measure unit of the one or more fuel sources. In some embodiments, the carbon intensity value comprises an amount of CO₂ emitted during the one or more process steps by the one or more fuel sources for an amount of each of the one or more raw materials. In some embodiments, the one or more raw materials comprise two or more raw materials. In some embodiments, the amount of CO2 emitted per measure unit of the one or more fuel sources includes an amount of CO2 emitted per a unit volume of the one or more fuel sources. In some embodiments, the amount of CO2 emitted per measure unit of the one or more fuel sources includes an amount of CO2 emitted per a unit mass of the one or more fuel sources. In some embodiments, the amount of CO2 emitted per measure unit of the one or more fuel sources include an amount of CO2 emitted per a unit weight of the one or more fuel sources. In some embodiments, the amount of CO2 emitted per measure unit of the one or more fuel sources include an amount of CO2 emitted per a unit energy of the one or more fuel sources.

In some embodiments, the one or more fuel sources are used to power one or more processing units associate with the one or more process steps. In some embodiments, the one or more processing units are configured to process at least a portion of a raw material input into a material output within the industrial process. In some embodiments, the system is configured to calculate the carbon footprint of industrial products (e.g., gasoline, diesel, and/or jet fuel) based on consumed processing energy at one or more steps from the raw material input to a final product output.

In some embodiments, the one or more non-transitory computer readable media further include program instructions stored thereon that when executed cause the one or more computers to determine, by the one or more processors, a carbon intensity value for each of the two or more raw materials. In some embodiments, a program step includes instructions to determine, by the one or more processors, which of the two or more raw materials comprises a lowest carbon intensity value. In some embodiments, a program step includes instructions to generate, by the one or more processors, a raw material purchase plan. In some embodiments, a program step includes instructions to display, by the one or more processors, the raw material purchase plan on the one or more GUIs.

In some embodiments, the raw material purchase plan includes a lowest carbon intensity raw material comprising at least one of the two or more raw materials with the lowest carbon intensity value. In some embodiments, the one or more fuel sources comprise two or more fuel sources. In some embodiments, the one or more non-transitory computer readable media further include program instructions stored thereon that when executed cause the one or more computers to receive, by the one or more processors, fuel source data comprising one or more of fuel source location data, fuel source type data, fuel source blend data, and fuel source property data for each of the two or more fuel sources. In some embodiments, a program step includes instructions to receive, by the one or more processors, one or more fuel source cost that includes historical, current, or future market cost for one or more fuel sources. In some embodiments, a program step includes instructions to determine, by the one or more processors, which of the two or more fuel sources comprises a lowest amount of CO₂ emitted per measure unit.

In some embodiments, a program step includes instructions to generate, by the one or more processors, a fuel source purchase plan. In some embodiments, a program step includes instructions to display, by the one or more processors, the fuel source purchase plan on the one or more GUIs. In some embodiments, the fuel source purchase plan includes a lowest emitting fuel source comprising at least one of the two or more fuel sources with the lowest amount of CO2 emitted per measurement unit.

In some embodiments, the one or more raw materials comprise one or more raw material blends. In some embodiments, each of the one or more raw material blends comprise two or more raw materials. In some embodiments, the one or more non-transitory computer readable media further include program instructions stored thereon that when executed cause the one or more computers to receive, by the one or more processors, an amount composition of each of the two or more raw materials for the one or more raw material blends. In some embodiments, a program step includes instructions to receive, by the one or more processors, the fuel source consumption data for each of the one or more raw material blends from the one or more sensors. In some embodiments, a program step includes instructions to store, by the one or more processors, the fuel source consumption data and the amount composition for the one or more raw material blends as historical blend data in the one or more non-transitory computer readable media.

In some embodiments, the one or more non-transitory computer readable media further include program instructions stored thereon that when executed cause the one or more computers to execute, by the one or more processors, a raw material blend analysis. In some embodiments, the raw material blend analysis comprises a blend carbon intensity prediction for the one or more process steps based at least in part on the historical blend data. In some embodiments, the blend carbon intensity prediction is based at least in part on the fuel emissions data. In some embodiments, the raw material blend analysis includes one or more blend predictions comprising a predicted lowest blend carbon intensity for a two or more raw material combination. In some embodiments, the two or more raw material combination includes an amount of each of the two or more raw materials.

In some embodiments, a program step includes instructions to execute, by the one or more processors, a carbon footprint analysis to determine a raw material blend combination that results in a lowest carbon footprint for the industrial process. In some embodiments, the one or more fuel sources comprise one or more fuel source blends each comprising two or more fuel sources. In some embodiments, the one or more non-transitory computer readable media further include program instructions stored thereon that when executed cause the one or more computers to execute, by the one or more processors, a fuel source blend analysis. In some embodiments, the fuel source blend analysis comprises a CO₂ prediction of the CO₂ emitted per measure unit of the one or more fuel sources.

In some embodiments, the one or more fuel source blends comprise at least one green fuel source. In some embodiments, the fuel source blend analysis comprises a green prediction. The green prediction comprises a reduction in net CO₂ emissions from the one or more fuel source blends. In some embodiments, the reduction is based at least in part on the amount of CO₂ removed from an atmosphere during a production of the green fuel source. In some embodiments, a green fuel source includes an energy source for powering the one or more process steps at least partially produced without a use of fossil fuels. In some embodiments, the at least one green fuel source includes an energy source derived from one or more of wind energy, solar energy, hydraulic energy, and biofuel. In some embodiments, a program step includes instructions to execute, by the one or more processors, a fuel blend analysis. In some embodiments, the fuel blend analysis includes a fuel combination of at least one fossil fuel source and the least one green fuel source that results in a lowest cost. In some embodiments, the lowest cost is based at least in part on a current and/or future cost of a green fuel source.

In some embodiments, a program step includes instructions to execute, by the one or more processors, a model simulation that includes one or more process steps models that each represent a respective one of the one or more process steps. In some embodiments, a program step includes instructions to determine, by the one or more processors, one or more process setpoints for the one or more process steps. In some embodiments, the system is configured to send one or more commands to one or more controllers based on the determined one or more setpoints. In some embodiments, the one or more controllers are configured to control the one or more process steps.

In some embodiments, a program step includes instructions to execute, by the one or more processors, one or more process changes that aligns with the one or more process setpoints. In some embodiments, a program step includes instructions to execute, by the one or more processors, one or more process changes that result in the delivery of the one or more fuel sources (e.g., fuel source blends) that align with the one or more process setpoints. In some embodiments, a program step includes instructions to execute, by the one or more processors, one or more process changes that result in the delivery of the one or more raw materials (e.g., raw material blends) that align with the one or more process setpoints. In some embodiments, a program step includes instructions to generate, by the one or more processors, a graphical user interface comprising a carbon intensity window. In some embodiments, the carbon intensity window comprises a visualization of the carbon intensity analysis. In some embodiments, a program step includes instructions to generate, by the one or more processors, a graphical user interface comprising a fuel source purchase window. In some embodiments, the fuel source purchase window comprises a visualization of the fuel source purchase analysis. In some embodiments, a program step includes instructions to generate, by the one or more processors, a graphical user interface comprising a raw material purchase window. In some embodiments, wherein the raw material purchase window comprises a visualization of the raw material purchase analysis.

### DRAWING DESCRIPTION

FIG. 1 illustrates a refinery model analysis GUI according to some embodiments.

FIG. 2 illustrates an output analysis GUI associated with a modeled component representing a physical component in a refinement process according to some embodiments.

FIG. 3 illustrates a Sankey model GUI including one or more modeled components and as well as the carbon intensity used to power the one or more modeled components according to some embodiments.

FIG. 4 shows further analysis by the system of the treated gasoil according to some embodiments.

FIG. 5 depicts the biggest contributors to the diesel pool in terms of carbon intensity according to some embodiments.

FIG. 6A illustrates a blend carbon intensity analysis according to some embodiments.

FIG. 6B illustrates a blend carbon intensity prediction according to some embodiments.

FIG. 7 illustrates a computer system enabling or comprising the systems and methods in accordance with some embodiments.

### DETAILED DESCRIPTION

In some embodiments, the system is configured to identify one or more properties of a crude material from a crude material source. In some embodiments, a crude material source includes one or more of a geological location, a well, a vendor, and a crude material reserve. In some embodiments, the system is configured to identify one or more properties of a fuel from a fuel source by analyzing the output of after a process step. In some embodiments, a fuel source (also referred to herein as a utility) includes a combustible material, a flammable material, a chemical, and/or an electrical energy source. In some embodiments, each crude material coming from its respective crude material source has its own properties. In some embodiments, the system is configured to identify one or more different properties in a crude material and/or a fuel and determine one or more refinery process setpoints to achieve a refined product with an optimal carbon footprint.

In some embodiments, the system is configured to analyze a refined product to determine one or more setpoint within a refinery process. It is understood that the systems and methods described herein are not limited to refinery processes and can be applied to any process. In some embodiments, the system is configured to send one or more commands to one or more refinery components (e.g., valves, distillation units, sulfur recovery unit, etc.) based on the determined setpoints. In some embodiments, the system is configured to decrease the carbon emissions of the final product and/or the refining step by determining a blend of different crude materials from different crude sources. In some embodiments, the system is configured to send one or more commands to one or more refinery components (e.g., valves, distillation units, sulfur recovery unit, etc.) to create the determined blend. In some embodiments, each blend requires one or more new setpoints for one or more components in the process. In some embodiments, the blends help to fight climate change as the least carbon intensive properties from each source are combined in a synergistic effect.

In some embodiments, the system is configured to create a purchase plan to obtain the optimum blends determined by the system. In some embodiments, the purchase plan includes one or more logistics such as receiving one or more crude material source scheduled outputs, transportation availability, storage availability, and/or refinement facility availability. In some embodiments, the system is configured to receive one or more properties of the crude from the different sources.

In some embodiments, the system is configured to improve CO₂ tracking capabilities by segregating total CO₂ refining emissions between “Fossil CO₂” and “Green CO₂” to account for introduction of renewable fuels in the refining process. In some embodiments, the system includes embedded optimization capabilities configured to reduce “Fossil CO₂” by executing sensitivity and/or scenario analysis. In some embodiments, executing sensitivity and/or scenario analysis includes generating one or more graphical user interfaces (GUIs) comprising one or more Sankey diagrams to visually trace CO₂ emissions flow across the refinery model.

In some embodiments, the system is configured to calculate the carbon emission pertaining to both intermediate and final refinery products. In some embodiments, these carbon emissions are due to utilities (i.e., fuel source) consumed by the processing unit and are in turn linked to carbon emissions pertaining to generation of the utilities. In some embodiments, the system is configured to classify the carbon emissions and hence the carbon intensity as green carbon or conventional carbon based on how the utilities are generated. In some embodiments, within a refinery, streams of raw material are fed through multiple process units. In some embodiments, each processing unit will contribute to a carbon intensity. In some embodiments, the system is configured to calculate the carbon intensity of the end products (like gasoline, diesel and jet) based on consumed processing energy at one or more steps from the crude material input to the final product output. In some embodiments, the system is configured to track the carbon intensity across the refinery and incorporate the results into a carbon optimization analysis and/or control feedback loop configured to lower carbon emissions. In some embodiments, the system is configured to execute a cost-benefit analysis to determine which utility, crude material source, and/or combination thereof results in one or more of the lowest carbon intensity and the lowest cost.

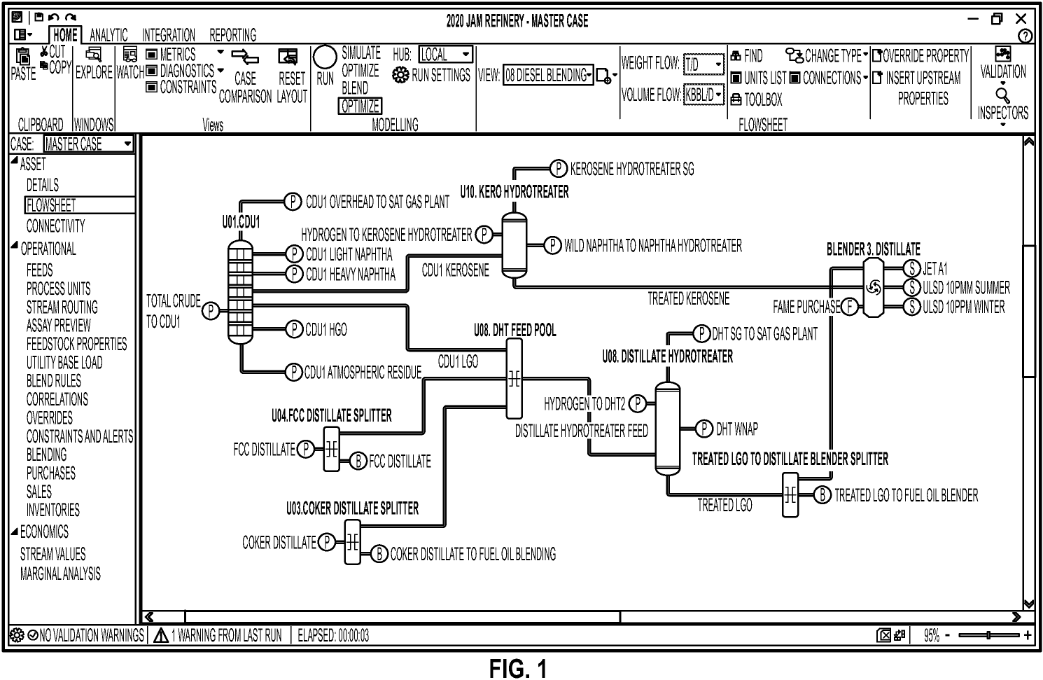

FIG. 1 illustrates a refinery model analysis window according to some embodiments. In some embodiments, the analysis window includes a simulation canvas for creating a model flowsheet as well as a model library. In some embodiments, the system is configured to enable a user to create and/or import one or more modeled components into the simulation canvas to create the flowsheet. In some embodiments, the system is configured to receive analytical data about the product output from one or more physical components and display the analysis in association with the one or more modeled components outputs.

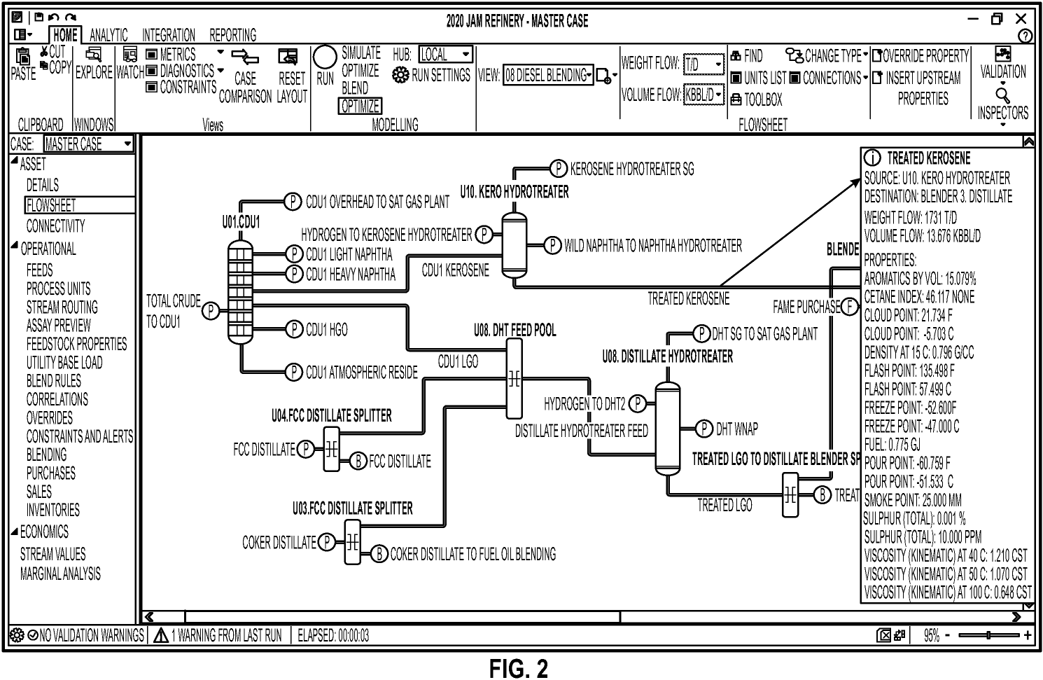

FIG. 2 illustrates an output analysis window associated with a modeled component representing a physical component in a refinement process. In some embodiments, the system is configured to display the analysis window with a GUI in response to a user selecting an output line connected to an output end of a modeled component. As shown, in some embodiments the analysis window comprises one or more properties of an output product from the physical component. In some embodiments, the system is configured to identify the origin of one or more contributors to carbon intensity for one or more steps, one or more process outputs, one or more refined products, and/or one or more final products. In some embodiments, the system provides insight into areas requiring additional CO₂ management that would otherwise be unobvious by reviewing the flowsheet of FIG. 1. In some embodiments, the system is configured to convert an object process model such as shown in FIGS. 1 and 2 into a Sankey process model in order to visualize the analysis of the carbon intensity contribution from one or more steps in the process.

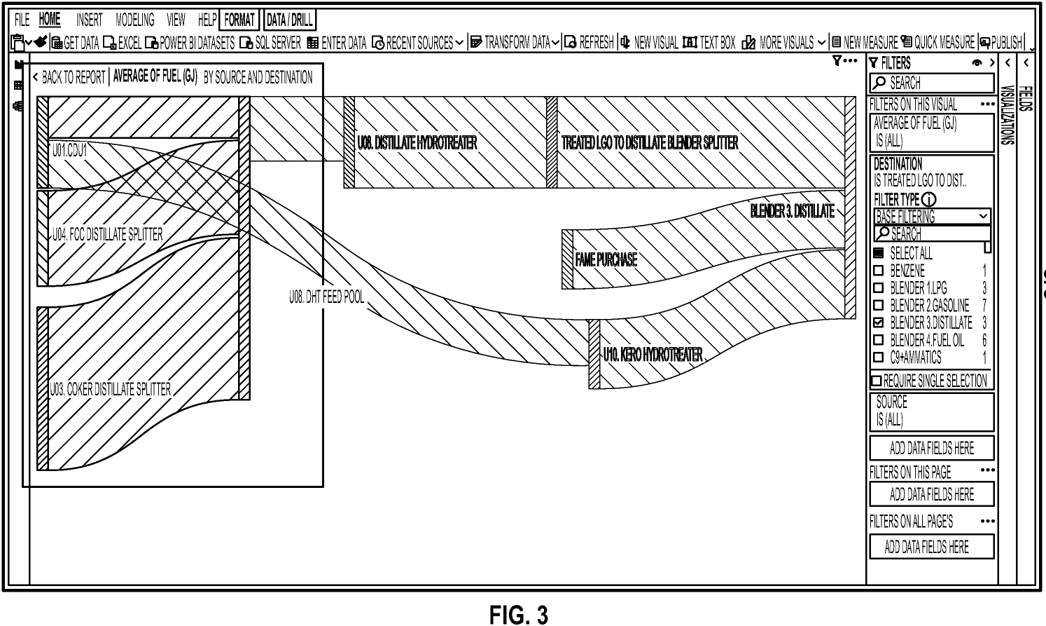

FIG. 3 illustrates a Sankey model including one or more modeled components and as well as the carbon intensity resulting from powering the one or more modeled components. In this example, the Sankey model shows three processing units (components) in a refinery process producing output products contributing to a diesel hydrotreating (DTH) feed pool according to some embodiments: a crude distillation unit (CDU), a fluid catalytic cracking (FCC) distillate splitter, and a coker distillate splitter.

In some embodiments, the thickness of each line corresponds to the carbon intensity of each step.

In some embodiments, carbon intensity includes the amount of CO₂ emissions produced by a given volume of a fuel source powering a processing unit for a given volume of output product. In some embodiments, the system is configured to receive fuel emission data for one or more fuel source types used in the process, where the fuel emission data comprises a carbon content value for determining the amount of CO₂ will generated (i.e., emitted) for a given volume of a fuel source. In some embodiments, the system is configured to assign carbon intensity to each output product from each physical component within the process, which in this case is the DHT feed pool. As evident from the Sankey model, the coker distillate splitter is the largest contributor to carbon intensity at this step.

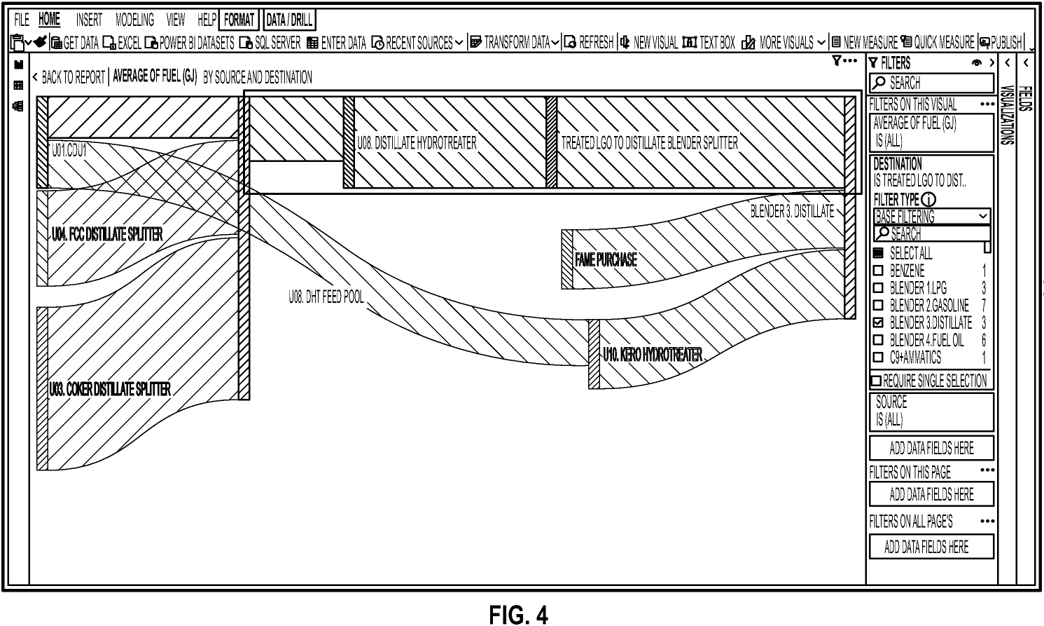

FIG. 4 shows further analysis by the system for the refinement of the DHT feed pool into treated gasoil. In some embodiments, the analysis shows that the distillate hydrotreater process is adding to the treated gasoil refinement carbon intensity for the blender distillate output while the distillate blender splitter itself does not contribute significantly to carbon intensity.

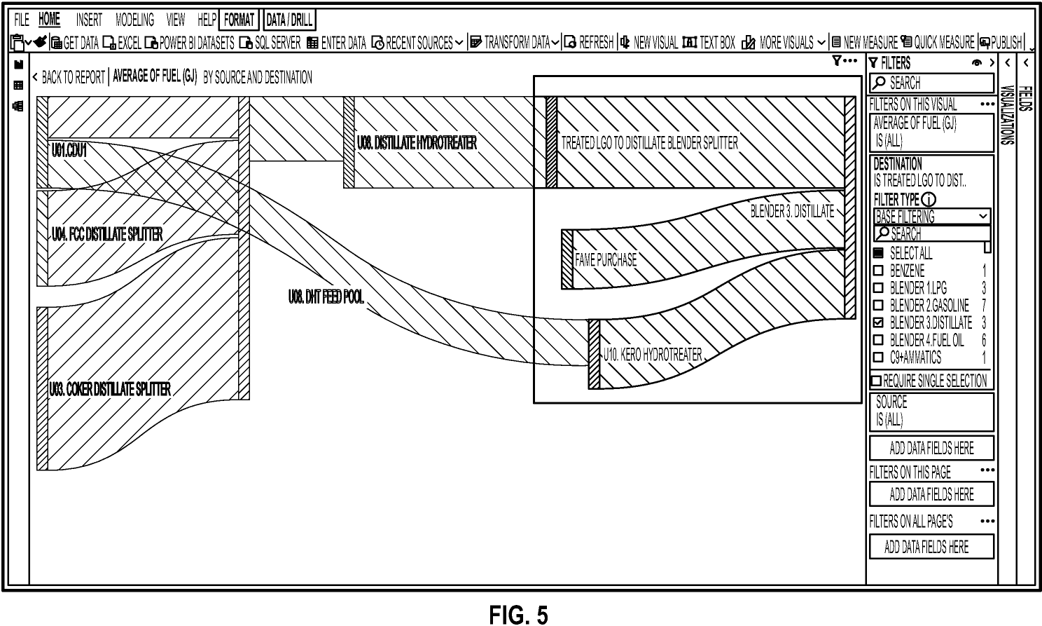

FIG. 5 shows that the additional carbon intensity at the blender distillate output for the treated gasoil refinement is a result of the addition of fatty acid methyl esters (FAME) as well as from the fuel source used for the kerosene (Kero) hydrotreater process as unrefined material is fed from the CDU according to some embodiments.

FIGS. 1 - 5 represent the carbon intensity for a single crude type from a single crude source according to some embodiments. However, in some embodiments, the crude material being processed includes a crude material blend. In some embodiments, the blend may be a type blend, a source blend, or some combination blend comprising one or more crude types and one or more crude sources. In some embodiments, differences in the chemical composition between different subsets of crude materials require different amounts of energy input to a process component for a given step. This changes the carbon intensity for a given product output for each step, which makes carbon intensity prediction for a step unfeasible without the benefits of the system described herein according to some embodiments. In some embodiments, predictions become even more complex when a crude material blend is used.

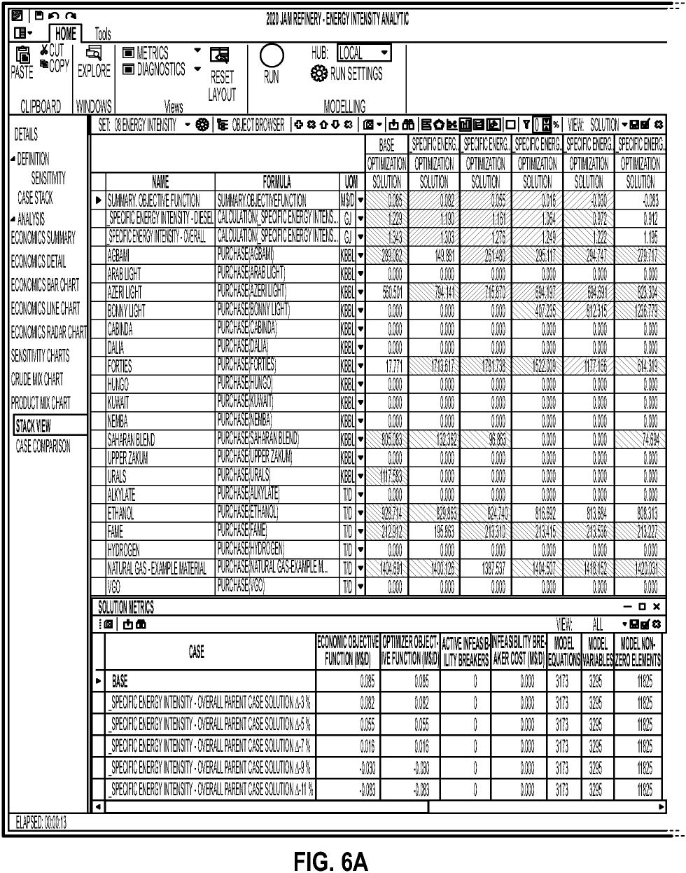

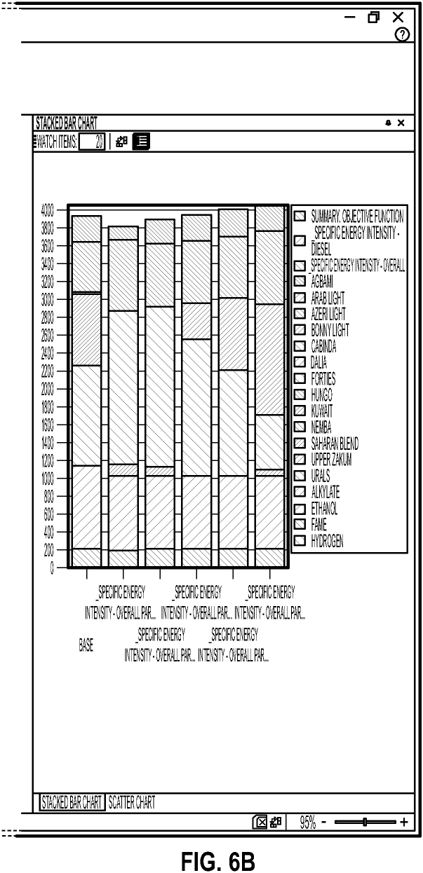

FIG. 6A illustrates a blend carbon intensity analysis according to some embodiments. FIG. 6B illustrates a blend carbon intensity prediction according to some embodiments. In some embodiments, the system is configured to use sample data obtained from one or more sensors to determine output product properties at one or more process outputs. In some embodiments, the system is configured to perform the analytics one each pipe in one or more refinement processes. In some embodiments, the system is configured to analyze one or more lines separately. In some embodiments, the system is configured to select and/or order one or more crude materials that result in the lowest carbon footprint representative of the total carbon intensity for all process units in an industrial process. In some embodiments, the system is configured to suggest a blend of crude material that results in the lowest carbon footprint. In some embodiments, the system is configured to suggest a blend of crude material that results in the lowest carbon intensity for one or more process steps.

In some embodiments, the system is configured to display one or more fuel sources options and or crude material options. In some embodiments, the system is configured to display one or more historical, current, or future market costs for one or more crude material options. In some embodiments, the system is configured to display one or more historical, current, or future market cost for one or more fuel sources. In some embodiments, the system is configured to output a suggest of one or more green fuels instead of carbon intensive fuels to lower the industrial processes carbon footprint. In some embodiments, the system is configured to execute a carbon analysis that outputs an amount of carbon intensity reduction per step and/or a carbon footprint for the industrial facility for a green fuel at a given fuel cost. As a non-limiting example, in some embodiments, a crude material or process output that requires more energy to operate can use green fuels in the respective processing steps to maintain a selected average cost while decreasing the carbon footprint.

In some embodiments, the system is configured to execute one or more control operations that result in an optimized blend of two or more crude materials into one or more steps of the process.

In some embodiments, the system comprises a scheduling module and an ordering module.

In some embodiments, the scheduling module is configured to schedule one or more process control operations that correlate with the suggested results of the carbon intensity analysis. In some embodiments, the ordering module is configured to order one or more crude materials and/or utility services that provide the lowest carbon footprint automatically. In some embodiments, the system is configured to automatically create and/or execute a purchase order for the crude that has the lowest carbon footprint and/or optimizes carbon footprint in view of other factors taken together.

In some embodiments, the system is configured to execute a validation step for ordering utilities and/or controlling the process. In some embodiments, the system is configured to display a validation window on a human-machine interface (HMI) and is configured to accept an input from a user confirming or denying the automatic execution of one or more system functions described herein.

In some embodiments, the system is configured to display a production plan based on the available crude supplies and execute a crude material switching operation according to the production plan. In some embodiments, the system is configured to automatically adjust the setpoints of one or more refinery components based on the production plan. In some embodiments, the system includes gets the information from the plant through a rigorous online modeling system and/or one or more outside online databases including one or more historian databases. In some embodiments, the analysis includes the composition of the material that is running through a pipe. In some embodiments, the system includes the use of artificial intelligence (AI) to determine the crude material mix to get the lowest carbon intensity and/or footprint.

In some embodiments, the system is configured to determine the properties of the blends. In some embodiments, the system is configured to execute a blend from one or more sources within the physical process. In some embodiments, the system is configured to suggest and/or one nor more components within the process based on the predictive properties of the mixture of the crude materials.

In some embodiments, the system is configured to translate one or more production plans into one or more execution steps implemented by the system. In some embodiments, one or more execution steps includes providing one or more setpoints to one or more programmable logic controller (PLCs) or other devices controlling one or more process components.

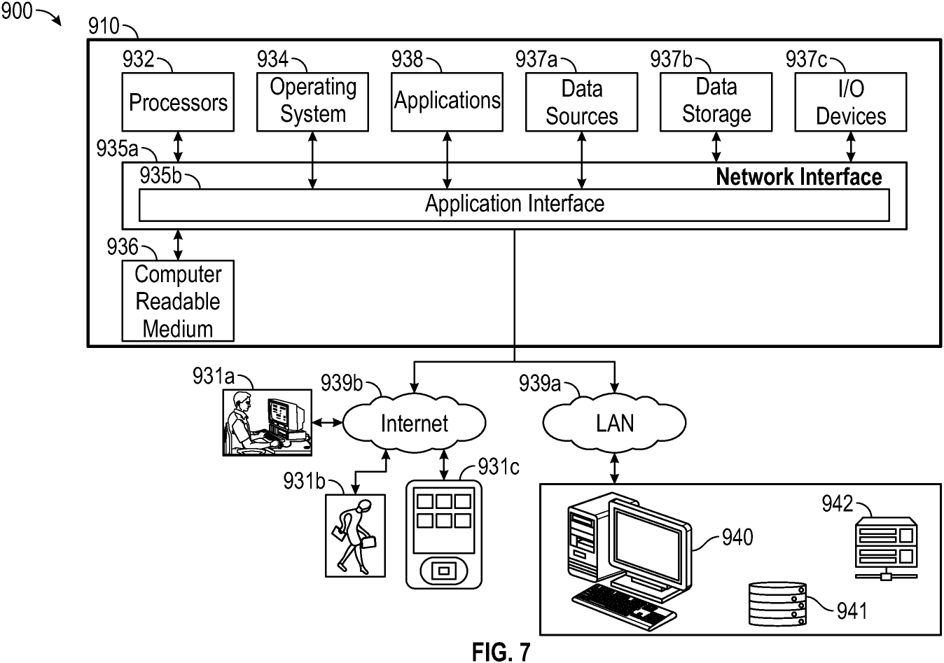

FIG. 7 illustrates a computer system 910 enabling or comprising the systems and methods in accordance with some embodiments of the system. In some embodiments, the computer system 910 can operate and/or process computer-executable code of one or more software modules of the aforementioned system and method. Further, in some embodiments, the computer system 910 can operate and/or display information within one or more graphical user interfaces (e.g., HMIs) integrated with or coupled to the system.

In some embodiments, the computer system 910 can comprise at least one processor 932. In some embodiments, the at least one processor 932 can reside in, or coupled to, one or more conventional server platforms (not shown). In some embodiments, the computer system 910 can include a network interface 935a and an application interface 935b coupled to the least one processor 932 capable of processing at least one operating system 934. Further, in some embodiments, the interfaces 935a, 935b coupled to at least one processor 932 can be configured to process one or more of the software modules (e.g., such as enterprise applications 938). In some embodiments, the software application modules 938 can include server-based software and can operate to host at least one user account and/or at least one client account, and operate to transfer data between one or more of these accounts using the at least one processor 932.

With the above embodiments in mind, it is understood that the system can employ various computer-implemented operations involving data stored in computer systems. Moreover, the above-described databases and models described throughout this disclosure can store analytical models and other data on computer-readable storage media within the computer system 910 and on computer-readable storage media coupled to the computer system 910 according to various embodiments. In addition, in some embodiments, the above-described applications of the system can be stored on computer-readable storage media within the computer system 910 and on computer-readable storage media coupled to the computer system 910. In some embodiments, these operations are those requiring physical manipulation of physical quantities. Usually, though not necessarily, in some embodiments these quantities take the form of one or more of electrical, electromagnetic, magnetic, optical, or magneto-optical signals capable of being stored, transferred, combined, compared and otherwise manipulated. In some embodiments, the computer system 910 can comprise at least one computer readable medium 936 coupled to at least one of at least one data source 937a, at least one data storage 937b, and/or at least one input/output 937c. In some embodiments, the computer system 910 can be embodied as computer readable code on a computer readable medium 936. In some embodiments, the computer readable medium 936 can be any data storage that can store data, which can thereafter be read by a computer (such as computer 940). In some embodiments, the computer readable medium 936 can be any physical or material medium that can be used to tangibly store the desired information or data or instructions and which can be accessed by a computer 940 or processor 932. In some embodiments, the computer readable medium 936 can include hard drives, network attached storage (NAS), read-only memory, random-access memory, FLASH based memory, CD-ROMs, CD-Rs, CD-RWs, DVDs, magnetic tapes, other optical and non-optical data storage. In some embodiments, various other forms of computer-readable media 936 can transmit or carry instructions to a remote computer 940 and/or at least one user 931, including a router, private or public network, or other transmission or channel, both wired and wireless. In some embodiments, the software application modules 938 can be configured to send and receive data from a database (e.g., from a computer readable medium 936 including data sources 937a and data storage 937b that can comprise a database), and data can be received by the software application modules 938 from at least one other source. In some embodiments, at least one of the software application modules 938 can be configured within the computer system 910 to output data to at least one user 931 via at least one graphical user interface rendered on at least one digital display.

In some embodiments, the computer readable medium 936 can be distributed over a conventional computer network via the network interface 935a where the system embodied by the computer readable code can be stored and executed in a distributed fashion. For example, in some embodiments, one or more components of the computer system 910 can be coupled to send and/or receive data through a local area network (“LAN”) 939a and/or an internet coupled network 939b (e.g., such as a wireless internet). In some embodiments, the networks 939a, 939b can include wide area networks (“WAN”), direct connections (e.g., through a universal serial bus port), or other forms of computer-readable media 936, or any combination thereof.

In some embodiments, components of the networks 939a, 939b can include any number of personal computers 940 which include for example desktop computers, and/or laptop computers, or any fixed, generally non-mobile internet appliances coupled through the LAN 939a. For example, some embodiments include one or more of personal computers 940, databases 941, and/or servers 942 coupled through the LAN 939a that can be configured for any type of user including an administrator. Some embodiments can include one or more personal computers 940 coupled through network 939b. In some embodiments, one or more components of the computer system 910 can be coupled to send or receive data through an internet network (e.g., such as network 939b). For example, some embodiments include at least one user 931a, 931b, is coupled wirelessly and accessing one or more software modules of the system including at least one enterprise application 938 via an input and output (“I/O”) 937c. In some embodiments, the computer system 910 can enable at least one user 931a, 931b, to be coupled to access enterprise applications 938 via an I/O 937c through LAN 939a. In some embodiments, the user 931 can comprise a user 931a coupled to the computer system 910 using a desktop computer, and/or laptop computers, or any fixed, generally non-mobile internet appliances coupled through the internet 939b. In some embodiments, the user can comprise a mobile user 931b coupled to the computer system 910. In some embodiments, the user 931b can connect using any mobile computing 931c to wireless coupled to the computer system 910, including, but not limited to, one or more personal digital assistants, at least one cellular phone, at least one mobile phone, at least one smart phone, at least one pager, at least one digital tablets, and/or at least one fixed or mobile internet appliances.

The subject matter described herein are directed to technological improvements to the field of process control by identifying raw materials and utility power suppliers that provide the lowest carbon intensity. The disclosure describes the specifics of how a machine including one or more computers comprising one or more processors and one or more non-transitory computer readable media implement the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Furthermore, acting as Applicant’s own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system for controlling a carbon footprint of an industrial process comprising:
one or more computers comprising one or more processors and one or more non-transitory computer readable media, the one or more non-transitory computer readable media including program instructions stored thereon that when executed cause the one or more computers to:
receive, by the one or more processors, raw material data comprising one or more of raw material location data, raw material type data, raw material blend data, and raw material property data for each of one or more raw materials;
monitor, by the one or more processors, one or more sensors configured to determine fuel source consumption data comprising an amount of one or more fuel sources required by one or more process steps in the industrial process;
determine, by the one or more processors, the amount of one or more fuel sources required by one or more process steps in the industrial process to process each of the one or more raw materials;
receive, by the one or more processors, fuel source data comprising fuel emissions data for the one or more fuel sources;
execute, by the one or more processors, a carbon intensity analysis configured to output a carbon intensity value for at least one of the one or more process steps based on the fuel emissions data;
generate, by the one or more processors, a carbon intensity report comprising the carbon intensity value; and
display, by the one or more processors, the carbon intensity report on one or more graphical user interfaces (GUIs);
wherein the fuel emissions data comprises an amount of CO₂ emitted per measure unit of the one or more fuel sources; and
wherein the carbon intensity value comprises an amount of CO₂ emitted during the one or more process steps by the one or more fuel sources for an amount of each of the one or more raw materials.

2. The system of claim 1,
wherein the one or more raw materials comprise two or more raw materials;
wherein the one or more non-transitory computer readable media further include program instructions stored thereon that when executed cause the one or more computers to:
determine, by the one or more processors, a carbon intensity value for each of the two or more raw materials;
determine, by the one or more processors, which of the two or more raw materials comprises a lowest carbon intensity value;
generate, by the one or more processors, a raw material purchase plan; and display, by the one or more processors, the raw material purchase plan on the one or more GUIs;
wherein the raw material purchase plan includes a lowest carbon intensity raw material comprising at least one of the two or more raw materials with the lowest carbon intensity value.

3. The system of claim 1,
wherein the one or more fuel sources comprise two or more fuel sources;
wherein the one or more non-transitory computer readable media further include program instructions stored thereon that when executed cause the one or more computers to:
receive, by the one or more processors, fuel source data comprising one or more of fuel source location data, fuel source type data, fuel source blend data, and fuel source property data for each of the two or more fuel sources;
determine, by the one or more processors, which of the two or more fuel sources comprises a lowest amount of CO₂ emitted per measure unit;
generate, by the one or more processors, a fuel source purchase plan; and
display, by the one or more processors, the fuel source purchase plan on the one or more GUIs;
wherein the fuel source purchase plan includes a lowest emitting fuel source comprising at least one of the two or more fuel sources with the lowest amount of CO₂ emitted per measurement unit.

4. The system of claim 1,
wherein the one or more raw materials comprise one or more raw material blends;
wherein each of the one or more raw material blends comprise two or more raw materials;
wherein the one or more non-transitory computer readable media further include program instructions stored thereon that when executed cause the one or more computers to:
receive, by the one or more processors, an amount composition of each of the two or more raw materials for the one or more raw material blends;
receive, by the one or more processors, the fuel source consumption data for each of the one or more raw material blends from the one or more sensors; and
store, by the one or more processors, the fuel source consumption data and the amount composition for the one or more raw material blends as historical blend data in the one or more non-transitory computer readable media.

5. The system of claim 4,
wherein the one or more non-transitory computer readable media further include program instructions stored thereon that when executed cause the one or more computers to:
execute, by the one or more processors, a raw material blend analysis;
wherein the raw material blend analysis comprises a blend carbon intensity prediction for the one or more process steps based at least in part on the historical blend data.

6. The system of claim 5,
wherein the blend carbon intensity prediction is based at least in part on the fuel emissions data.

7. The system of claim 5,
wherein the raw material blend analysis includes one or more blend predictions comprising a predicted lowest blend carbon intensity for a two or more raw material combination; and
wherein the two or more raw material combination includes an amount of each of the two or more raw materials.

8. The system of claim 5,
wherein the one or more non-transitory computer readable media further include program instructions stored thereon that when executed cause the one or more computers to:
execute, by the one or more processors, a carbon footprint analysis to determine a raw material blend combination that results in a lowest carbon footprint for the industrial process.

9. The system of claim 5,
wherein the one or more fuel sources comprise one or more fuel source blends each comprising two or more fuel sources; and
wherein the one or more non-transitory computer readable media further include program instructions stored thereon that when executed cause the one or more computers to:
execute, by the one or more processors, a fuel source blend analysis;
wherein the fuel source blend analysis comprises a CO₂ prediction of the CO₂ emitted per measure unit of the one or more fuel sources.

10. The system of claim 9,
wherein the one or more fuel source blends comprise at least one green fuel source;
wherein the fuel source blend analysis comprises a green prediction; and
wherein the green prediction comprises a reduction in net CO₂ emissions from the one or more fuel source blends; and
wherein the reduction is based at least in part on the amount of CO₂ removed from an atmosphere during a production of the green fuel source.

11. The system of claim 10,
wherein a green fuel source includes an energy source for powering the one or more process steps at least partially produced without a use of fossil fuels.

12. The system of claim 10,
wherein the at least one green fuel source includes an energy source derived from one or more of wind energy, solar energy, hydraulic energy, and biofuel.

13. The system of claim 10,
wherein the one or more non-transitory computer readable media further include program instructions stored thereon that when executed cause the one or more computers to:
execute, by the one or more processors, a fuel blend analysis;
wherein the fuel blend analysis includes a fuel combination of at least one fossil fuel source and the least one green fuel source that results in a lowest cost; and
wherein the lowest cost is based at least in part on a current and/or future cost of a green fuel source.

14. The system of claim 1,
wherein the one or more non-transitory computer readable media further include program instructions stored thereon that when executed cause the one or more computers to:
execute, by the one or more processors, a model simulation that includes one or more process steps models that each represent a respective one of the one or more process steps.

15. The system of claim 14,
wherein the one or more non-transitory computer readable media further include program instructions stored thereon that when executed cause the one or more computers to:
determine, by the one or more processors, one or more process setpoints for the one or more process steps;
wherein the system is configured to send one or more commands to one or more controllers based on the determined one or more setpoints; and
wherein the one or more controllers are configured to control the one or more process steps.
